(19) United States
(12) Patent Application Publication
(10) Pub. No.: US 2014/0094809 A1
(43) Pub. Date: Apr. 3, 2014

(54) METHOD OF REDUCING SIDEWALL COMPACTION IN A BONE OR ARTICULAR JOINT PENETRATION

(71) Applicants: Lanny L. Johnson, Frankfort, MI (US); Myron Spector, Boston, MA (US)

(72) Inventors: Lanny L. Johnson, Frankfort, MI (US); Myron Spector, Boston, MA (US)

(21) Appl. No.: 14/096,832

(22) Filed: Dec. 4, 2013

Related U.S. Application Data
(62) Division of application No. 13/633,872, filed on Oct. 2, 2012.

(51) Int. Cl. A61B 17/16

(52) U.S. Cl. CPC A61B 17/1659
USPC 606/80

(57) ABSTRACT

A method of creating a penetration with reduced sidewall compaction in a surface of a bone or articular joint. The method includes the steps of positioning a surgical instrument in contact with the surface, applying a longitudinal force to the surgical instrument causing the surgical instrument to penetrate the surface thereby creating a penetration with at least one sidewall, and retracting the surgical instrument and strategically scraping a portion of the surgical instrument against a portion of the at least one sidewall to remove compacted material from the penetration, thereby creating a small surgically created wound in the surface having reduced compaction at the margins to promote rapid vascular penetration, cellular migration, and faster healing with an enhanced histologically confirmed maturing tissue. The surgical defect can be round or slit-like.

### CROSS REFERENCE TO RELATED APPLICATIONS

This application is a divisional application of U.S. nonprovisional application Ser. No. 13/633,872, filed on Oct. 2, 2012, all of which is incorporated by reference as if completely written herein.

### BACKGROUND OF THE INVENTION

The present invention relates to surgical instruments and, more particularly, to a surgical instrument for penetrating bone or an articular joint surface such a manner that, upon removal of the instrument, bone and or cartilage remnants are removed from the sides of the defect so as to avoid leaving compacted bone in the miniature precise defect.

Existing surgical instruments used for penetration into subchondral bone often results in a non-precise defect of relatively large size, pushing bone to the side, resulting in a thickened compacted bony wall that resists or delays vascular penetration into the defect and resulting healing.

Existing awl-type instruments typically used for this purpose are round, tapered with straight, and 45 degree angle, slanted configuration. They have smooth sides that, when penetrating bone, push bone or cartilage to the side of the defect resulting in the bone being compacted to the side and against the walls of the surgical defect.

The existing instruments' geometry allows only a circular awl configuration. Therefore they create large defects with compacted bone on the walls. They do not remove bone from the walls when removed from the defect and, because of their geometry, they cannot create a linear defect and can only have an angle of attack or approach to a bony or articular surface at zero to perhaps 35 (+/−) degrees. When the latter is used, the result can be a much larger defect since the instrument “walks” or moves forward upon impaction.

As can be seen, there is a need for an improved surgical instrument for penetrating bone such that, upon removal, the instrument brings out bone and or cartilage with it to avoid leaving a barrier of compacted bone or tissue in the defect, thus creating a bony defect environment providing for good vascular penetration into the defect and resultant enhanced healing.

### SUMMARY OF INVENTION

In one aspect of the present invention, a surgical instrument comprises a main shaft; a plurality of ridges cut along the main shaft, the ridges including a concave surface and concave surface sharp edges; and a sharp tip disposed on a distal end of the main shaft.

In another aspect of the present invention, a method for penetrating bone in a surgical procedure comprises inserting a surgical instrument into bone or an articular joint surface to create a defect, the surgical instrument including a main shaft, a plurality of ridges cut along the main shaft, the ridges including a concave surface and concave surface sharp edges, and a sharp tip disposed on a distal end of the main shaft; and removing the surgical instrument from the defect where the concave surface sharp edges remove compacted bone and tissue from along a sides of the defect and the concave surface collects the compacted bone as the surgical instrument is removed.

These and other features, aspects and advantages of the present invention will become better understood with reference to the following drawings, description and claims.

### BRIEF DESCRIPTION OF THE DRAWINGS

FIG. 1 is a perspective view of a bone penetrating surgical instrument according to an exemplary embodiment of the present invention;

FIG. 2 is a perspective view of the bone penetrating surgical instrument of FIG. 1;

FIG. 3 is a cross-sectional view taken along line 3-3 of FIG. 2;

FIG. 4 is a perspective view of a bone penetrating surgical instrument according to another exemplary embodiment of the present invention;

FIG. 5 is a side view of the bone penetrating surgical instrument of FIG. 4; and

FIG. 6 is a front view of the bone penetrating surgical instrument of FIG. 4.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a surgical instrument in a sharp miniature chisel or awl shape that has a series of ridges along the course of the cutting tool. Each ridge has, on its proximal surface, a concave surface. Upon retraction, the instrument is pushed and or twisted against the side of the defect, allowing the compacted bone on the walls to be captured in the concave recesses of the instrument. Unlike existing instruments that can compact bone against the walls of the surgical defect, the present invention removes the compacted bone from the sides of the surgical defect, improving resultant vascular penetration and healing. The creation of a small surgically created wound in subchondral bone, absent the thickened compacted bone at the margins, responds with rapid vascular penetration/migration and faster healing with an enhanced histologically confirmed maturing tissue. The surgical defect can be round or slit-like. The instrument allows a wide spectrum of angle of attack to create the defect. Slit-type defects in subchondral bone typically heal faster with more mature cartilage tissue.

With the awl and chisel geometry, various angles of approaches are possible in the restricted intra-articular surgical spaces. Moreover, the more desirable slit-like defect is possible in most of the surgical exposures. No bone is compacted with the instrument of the present invention, but is, instead, removed retrograde by the concave surfaces surrounding the tool.

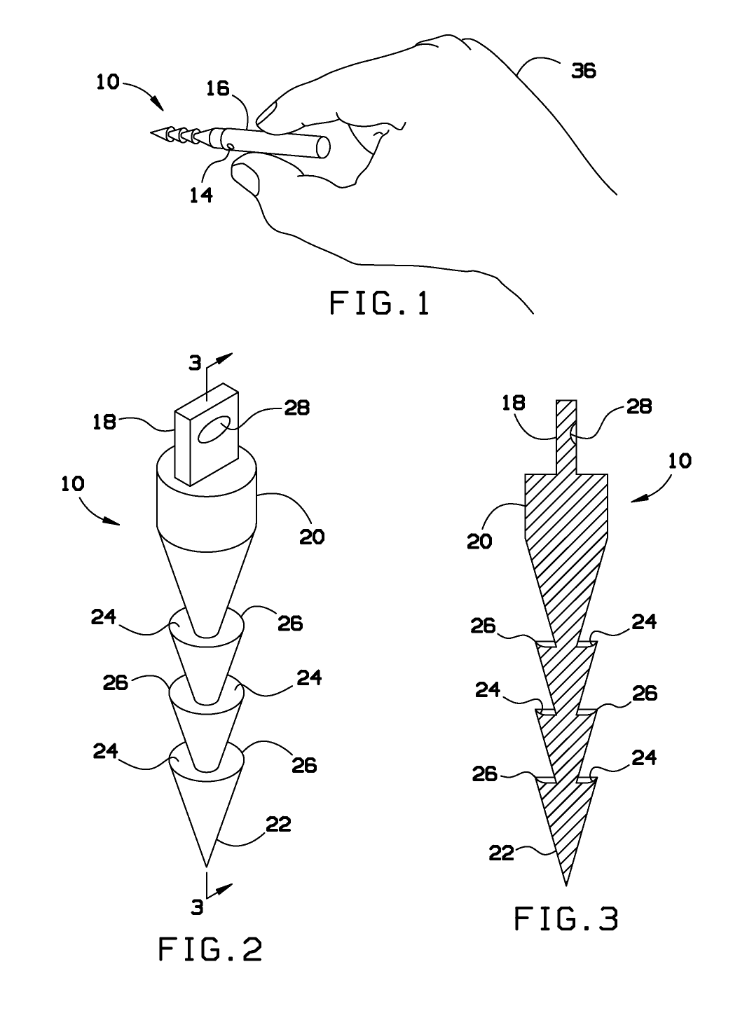

Referring now to FIGS. 1 through 3, an awl-type surgical instrument 10 can be disposed in a hand piece 16, secured with a set screw 14 that can engage with a set screw indention 28 in a hand piece mounting tab 18 of the surgical instrument 10. The hand piece 16 permits a user's hand 36 to hold and control the surgical instrument 10 via the hand piece or handle 16 providing opportunity for use of a hammer or mallet to forcefully create the surgical defect.

An awl main shaft 20 can include a sharp enough tip 22 to initiate penetration into the bone. A series of ridges can be cut along the main shaft 20 between the sharp tip 22 and the hand piece mounting tab 18. Any number of ridges can be disposed along the main shaft 20. Typically from one to five or more ridges may be disposed along the main shaft 20. The ridges can include concave surface sharp edges 26 with concave surfaces 24 formed on each ridge. Each ridge can be formed in a frusto-conical shape. FIG. 3 best shows the concave shape of the concave surfaces 24. The concave surfaces 24 allow the capture of the compacted bone on the walls when the surgical instrument 10 is removed or retracted.

The awl may be conical or angular. The recessed areas to collect bone may be vertical or horizontally oriented depending upon the geometry of the awl surgical instrument. The chisel may have projections on one or two sides for bone collection. Single-sided projections/ridges could be used in small joints to minimize the surgically created defect. In some embodiments, the awl surgical instrument

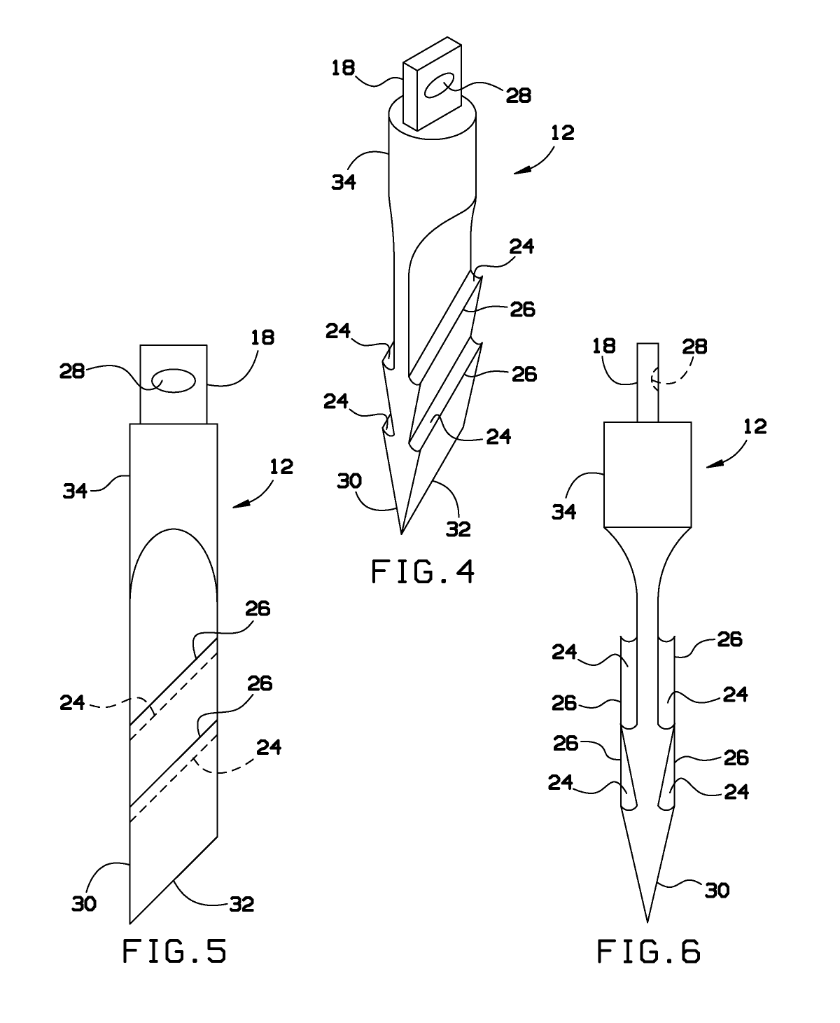

Referring now to FIGS. 4 through 6, a chisel-type surgical instrument 12 can be disposed in the hand piece 16, secured with the set screw or similar secure method of fixation 14 that can engage with the set screw indention 28 in the hand piece mounting tab 18 of the surgical instrument 12, similar to the awl-type surgical instrument 10 described above. The hand piece 16 permits the user's hand 36 to hold and control the surgical instrument 12 via the hand piece 16 including being of such a configuration to provide for impaction with a mallet or hammer.

A chisel main shaft 34 can include a chisel sharp tip 32. A series of ridges can be cut along the main shaft 34 between the chisel sharp tip 32 and the hand piece mounting tab 18. Any number of ridges can be disposed along the main shaft 34. Typically from one to five or more ridges may be disposed along the main shaft 34. The ridges can include concave surface sharp edges 26 with concave surfaces 24 formed on each ridge. As shown in FIG. 5, the concave surface sharp edges 26 can be disposed at an angle offset from perpendicular from a side edge of the surgical instrument 12. FIG. 6 best shows the concave shape of the concave surfaces 24. The concave surfaces 24 allow the capture of the compacted bone on the walls when the surgical instrument 12 is removed or retracted.

The surgical instruments 10, 12 of the present invention can be made of various materials by various processes, such as by a computer numerical controlled (CNC) machining process. They may be constructed in such a manner that the hand piece is contiguous with the cutting instrument.

The connecting hand piece material may be stainless steel, radel and phenolic. The connection, if not contiguous, is such that rotation is controlled and there is no loosening from the cutting tool upon removal, retraction, or manipulation. This connecting hand piece may be such that provision is made for hammering in and or out.

While the above describes hand tools, using the hand piece 16, the surgical instruments 10, 12 of the present invention could be mechanized, motor driven or other energy sources, is possible or feasible, depending on the application.

It should be understood, of course, that the foregoing relates to exemplary embodiments of the invention and that modifications may be made without departing from the spirit and scope of the invention as set forth in the following claims.

We claim:

1. A method of creating a penetration with reduced sidewall compaction in a surface of a bone or articular joint, the method comprising the steps of:
(a) positioning a surgical instrument in contact with the surface, wherein the surgical instrument has a shaft with a longitudinal axis terminating in a sharp tip, and at least one ridge on an exterior surface of the surgical instrument forming a sharp ridge edge;
(b) applying a longitudinal force to the surgical instrument causing the surgical instrument to penetrate the surface to a depth such that the at least one ridge is below the surface, thereby creating a penetration with at least one sidewall; and
(c) retracting the surgical instrument and scraping the at least one ridge against a portion of the at least one sidewall to remove compacted material from the penetration.

2. The method of claim 1, wherein the at least one ridge has a concave surface that opens away from the sharp tip and contacts at least a portion of the sharp ridge edge, and the step of scraping a portion of the at least one sidewall with the at least one ridge utilizes the concave surface to collect the compacted material to be removed during retraction of the surgical instrument.

3. The method of claim 2, wherein the at least one ridge and the concave surface extend circumferentially at least 25% of the way around the surgical instrument, and wherein the step of retracting the surgical instrument is performed with the longitudinal axis at a different orientation then the orientation of the longitudinal axis when the penetration is created.

4. The method of claim 3, wherein the at least one ridge and the concave surface extend circumferentially at least 50% of the way around the surgical instrument, and wherein the step of retracting the surgical instrument is performed with the longitudinal axis at a different orientation then the orientation of the longitudinal axis when the penetration is created.

5. The method of claim 4, wherein the at least one ridge and the concave surface extend circumferentially all the way around the surgical instrument, and wherein the step of retracting the surgical instrument is performed with the longitudinal axis at a different orientation then the orientation of the longitudinal axis when the penetration is created.

6. The method of claim 2, wherein the at least one ridge and the concave surface extend circumferentially at least 25% of the way around the surgical instrument in a single plane perpendicular to the longitudinal axis, and wherein the step of retracting the surgical instrument is performed with the longitudinal axis at a different orientation then the orientation of the longitudinal axis when the penetration is created.

7. The method of claim 6, wherein the at least one ridge and the concave surface extend circumferentially at least 50% of the way around the surgical instrument in a single plane perpendicular to the longitudinal axis, and wherein the step of retracting the surgical instrument is performed with the longitudinal axis at a different orientation then the orientation of the longitudinal axis when the penetration is created.

8. The method of claim 7, wherein the at least one ridge and the concave surface extend circumferentially all the way around the surgical instrument in a single plane perpendicular to the longitudinal axis, and wherein the step of retracting the surgical instrument is performed with the longitudinal axis at a different orientation then the orientation of the longitudinal axis when the penetration is created.

9. The method of claim 2, wherein the step of creating a penetration with at least one sidewall further creates a slit-like penetration having two sidewalls formed by the surgical instrument further having at least a portion with a rectangular cross-sectional shape in a plane perpendicular to the longitudinal axis, wherein at least a portion of the sharp ridge edge is linear along a first long side of the rectangular cross-sectional shape.

10. The method of claim 9, wherein at least a portion of the sharp ridge edge is linear along a second long side of the rectangular cross-sectional shape.

11. The method of claim 10, wherein the linear sharp ridge edge along the second long side of the rectangular cross-sectional shape is further from the sharp tip along the longitudinal axis than the linear sharp ridge edge along the first long side of the rectangular cross-sectional shape.

12. The method of claim 10, wherein the distance of linear sharp ridge edge along the second long side of the rectangular cross-sectional shape from the sharp tip along the longitudinal axis is equal to the distance of the linear sharp ridge edge along the first long side of the rectangular cross-sectional shape from the sharp tip along the longitudinal axis.

13. The method of claim 9, wherein the step of scraping a portion of the at least one sidewall with the at least one ridge includes the step of twisting the shaft about the longitudinal axis while retracting the surgical instrument so that a portion of the sharp ridge edge is forced against one of the sidewalls of the slit-like penetration.

14. The method of claim 10, wherein the step of scraping a portion of the at least one sidewall with the at least one ridge includes the step of twisting the shaft about the longitudinal axis while retracting the surgical instrument so that a portion of the sharp ridge edge along the first long side of the rectangular cross-sectional shape is forced against one of the sidewalls of the slit-like penetration, and a portion of the sharp ridge edge along the second long side of the rectangular cross-sectional shape is forced against a different one of the sidewalls of the slit-like penetration.

15. The method of claim 14, wherein the sharp ridge edge along the first long side of the rectangular cross-sectional shape is parallel to the sharp ridge edge along the second long side of the rectangular cross-sectional shape.

16. The method of claim 1, wherein the surgical instrument has a cross-sectional shape in a plane perpendicular to the longitudinal axis and at the point of maximum penetration no portion of the at least one ridge extends beyond the boundary of the largest cross-sectional shape of the surgical instrument below the surface thereby ensuring smooth retraction of the surgical instrument from the penetration when the longitudinal axis is in the same orientation as the longitudinal axis during penetration.

17. A method of creating a penetration with reduced sidewall compaction in a surface of a bone or articular joint, the method comprising the steps of:
(a) positioning a surgical instrument in contact with the surface, wherein the surgical instrument has:
(i) a shaft with a longitudinal axis terminating in a sharp tip;
(ii) at least one ridge on an exterior surface of the surgical instrument forming a sharp ridge edge;
(iii) a concave surface formed in the at least one ridge with the concave surface opening away from the sharp tip and contacting at least a portion of the sharp ridge edge; and
(iv) at least a portion with a rectangular cross-sectional shape in a plane perpendicular to the longitudinal axis wherein at least a portion of the sharp ridge edge is linear along a first long side of the rectangular cross-sectional shape;
(b) applying a longitudinal force to the surgical instrument causing the surgical instrument to penetrate the surface to a depth such that the at least one ridge is below the surface, thereby creating a slit-like penetration with at least two sidewalls; and
(c) retracting the surgical instrument and scraping the at least one ridge against a portion of the at least one sidewall to remove compacted material from the penetration, wherein the concave surface collects the compacted material to be removed during retraction of the surgical instrument, wherein the surgical instrument has a cross-sectional shape in a plane perpendicular to the longitudinal axis and at the point of maximum penetration no portion of the at least one ridge extends beyond the boundary of the largest cross-sectional shape of the surgical instrument below the surface thereby ensuring smooth retraction of the surgical instrument from the penetration when the longitudinal axis is in the same orientation as the longitudinal axis during penetration.

18. The method of claim 17, wherein at least a portion of the sharp ridge edge is linear along a second long side of the rectangular cross-sectional shape.

19. The method of claim 18, wherein the linear sharp ridge edge along the second long side of the rectangular cross-sectional shape is further from the sharp tip along the longitudinal axis than the linear sharp ridge edge along the first long side of the rectangular cross-sectional shape.

20. The method of claim 18, wherein the distance of linear sharp ridge edge along the second long side of the rectangular cross-sectional shape from the sharp tip along the longitudinal axis is equal to the distance of the linear sharp ridge edge along the first long side of the rectangular cross-sectional shape from the sharp tip along the longitudinal axis.
